(19) United States
(12) Patent Application Publication
(10) Pub. No.: US 2006/0229550 A1
(43) Pub. Date: Oct. 12, 2006

(54) Liquid jet surgical instrument

(75) Inventors: Kevin P. Staid, Lowell, MA (US); James J. Frassica, Chelmsford, MA (US); Brian G. Connor, Newfields, NH (US); Derek Bruce Eldridge, North Chelmsford, MA (US); Ernie Dion, Danvers, MA (US); James E. Barrington, Lexington, MA (US)

Correspondence Address: WOLF GREENFIELD & SACKS, PC, FEDERAL RESERVE PLAZA, 600 ATLANTIC AVENUE, BOSTON, MA, 02210-2206, US

(73) Assignee: HydroCision, Incorporated, Billerica, MA (US)

(21) Appl. No.: 11/303,461

(22) Filed: Dec. 14, 2005

Related U.S. Application Data
(60) Provisional application No. 60/636,421, filed on Dec. 14, 2004.

(51) Int. Cl. A61M 1/00

(52) U.S. Cl. 604/27

(57) ABSTRACT

The invention provides a variety of surgical instruments for forming a liquid jet, which are useful for performing a wide variety of surgical procedures. In some embodiments, the invention provides surgical liquid jet instruments having a pressure tube and an evacuation tube, where the pressure tube includes at least one nozzle for forming a liquid jet and where the evacuation tube includes a jet-receiving opening for receiving the liquid jet when the instrument is in operation. In some embodiments, the surgical liquid jet instrument is constructed to minimize damage to the tissue surrounding the tissue that is desired to be removed. In some embodiments, the outer surface of the distal tip of the evacuation tube wall and/or the pressure tube wall is blunted to minimize tissue damage. The invention also provides surgical methods utilizing the inventive surgical liquid jet instruments for cutting or ablating a selected tissue within portions of a patient's spine, such as within the intervertebral disc.

### RELATED APPLICATIONS

This application claims priority under 35 U.S.C. § 119(e) to U.S. Provisional Application Ser. No. 60/636,421 entitled “Fluid Jet Surgical Instruments,” filed on Dec. 14, 2004, which is herein incorporated by reference in its entirety.

### FIELD OF THE INVENTION

The invention relates generally to surgical instruments for creating a liquid jet and methods for using the instruments in surgical procedures.

### BACKGROUND OF THE INVENTION

Traditionally, many surgical procedures have been performed on patients using open surgical methods that utilize relatively large incisions to expose a surgical field. Many traditional methods have also typically utilized surgical tools such as scalpels, scrapers, blunt dissectors, lasers, electrosurgical devices, etc., which have poor tissue differentiating capability and which can easily cause inadvertent damage to tissue surrounding a surgical treatment site unless carefully utilized. Open surgery with such prior art surgical instruments often involves extensive trauma to the patient, with associated problems of long recovery periods and potential complications.

There has been a trend in recent years to perform many surgical procedures using less invasive techniques by accessing surgical sites via small holes through the skin or through body orifices. These techniques are known as “minimally invasive surgery.” Minimally invasive surgical techniques commonly employed include endoscopic, laparoscopic, and arthroscopic surgical procedures. Minimally invasive surgical procedures are commonly preferred to open surgical procedures for many applications because the minimally invasive procedures induce less trauma to the patient during surgery and involve, in many cases, fewer potential complications and reduced recovery time.

A variety of instruments have been developed and utilized for minimally invasive surgical procedures. Frequently used instruments include blades and scalpel-type instruments, motorized rotary blade instruments, laser instruments, and electrosurgical or electrocautery instruments. Typically, these prior art instruments suffer from a variety of disadvantages. For example, the instruments can be slow and laborious to use, typically they lack the ability to selectively differentiate tissue to be excised from non-target tissue, they tend to have sizes and/or shapes which make access of many surgical sites difficult, and they tend to cause unintended damage to tissue surrounding the intended target tissue. Most prior art instruments also require the operator to manually remove excised tissue, for example with forceps, or require an external source of vacuum to be applied to the surgical site, for example, via an aspiration tube that is separate from the surgical instrument, in order to remove excised tissue. For many minimally invasive surgical applications such as arthroscopy, certain spinal procedures etc., where visualization of the surgical site is typically effected using an imaging system having a probe such as a fiber optic probe inserted into the surgical site, the above mentioned prior art surgical instruments also typically make it difficult to clearly visualize the site of tissue excision within the surgical field by not effectively evacuating tissue and debris from the surgical site.

Instruments that employ liquid jets have also been utilized in surgical procedures for cutting and ablating tissue. Such instruments have many advantages over the above mentioned surgical instruments for performing both open and minimally invasive surgical procedures. For example, liquid jet instruments can avoid the thermal damage to surrounding tissues that is often caused by instruments such as lasers and electrosurgical devices. In recent years, liquid jet instruments have been utilized for a variety of surgical procedures including open surgical procedures such as liver resection, endoscopic procedures such as kidney stone disruption and removal, and arthrectomy procedures for removal of thrombotic tissue from the vascular system.

A variety of liquid jet instruments for surgery have been developed, including instruments described in commonly-owned U.S. Pat. No. 5,944,688, U.S. Pat. No. 6,375,635, U.S. Pat. No. 6,511,493, U.S. Pat. No. 6,451,017, U.S. Application Publication No. US2002-0177802, U.S. Application Publication No. US2002-0111579, U.S. Application Publication No. US2003-0125660, U.S. Application Publication No. US2002-0176788, U.S. Application Publication No. US2004-0228736, U.S. Application Publication No. US2004-0243157, and International Application Publication No. 2004/069064, which are incorporated by reference in their entirety. These surgical liquid jet cutting systems typically have a pump for pressurizing a liquid, such as isotonic saline or other physiologically-compatible liquid. The pressurized liquid is conveyed, for example by flexible tubing, to a handpiece which has a handle region, and a distal end configured to perform a surgical or medical procedure on a patient. The distal end of the instrument typically has a pressurizable pressure tube providing a lumen for conveying the pressurized liquid, and a nozzle, through which the pressurized liquid exits to form a liquid jet. These instruments may include an evacuation tube providing an evacuation lumen, which receives some or all of the liquid from the jet, as well as excised tissue, and removes such materials for disposal. The evacuation tube may have a diameter considerably larger than the diameter of the pressure tube. In some of these instruments, the jet is emitted “proximally”, i.e., in a direction back towards the handle. In other configurations, the jet may be emitted “laterally”, i.e. in a direction substantially perpendicular to the longitudinal axis of the pressure tube in regions proximal to the distal end of the instrument, “distally”, or at some intermediate angle.

While currently available surgical liquid jet instruments represent, in some instances, significant improvements over many prior art surgical instruments for performing open and minimally invasive surgical procedures, there remains a need in the art to provide liquid jet surgical instruments which have certain improved capabilities, and which have the ability to be utilized in a wide variety of open and minimally invasive surgical procedures. The present invention provides, in many embodiments, such improved surgical liquid jet instruments, and further provides methods for their use in a variety of surgical procedures.

There further remains a need in the art for a liquid jet surgical instrument which minimizes the trauma to the tissue surrounding the excised tissue. Although certain conventional surgical liquid jet instruments may be capable of cutting or ablating and/or removing tissue in a desired surgical area, they may not be designed to restrict tissue damage or removal from the adjacent healthy tissue regions for specific surgical procedures. Consequently, many of the prior surgical liquid jet instruments may have a tendency to inadvertently cut, ablate, and/or damage tissue regions surrounding a target tissue. This may lead to further scarring, additional pain, and further recovery time.

For example, the walls of the pressure and/or evacuation tubes of certain conventional liquid jet surgical instruments may be thin to enable them to be sufficiently small to fit into tight surgical spaces. Such a thin tube wall, e.g. one having a thickness of about 0.005 inch (0.125 mm), may be sharp. Thus, the action of moving an instrument with such thin tubing within a confined space may result in the inadvertent cutting of or damage to tissue other than the intended target, through the cutting action of a thin-walled component on the surgical instrument. In some procedures, providing a cutting edge as well as a liquid jet component may be desirable, as described in our copending U.S. Application Publication No. US2004-0243157. In other circumstances, however, this may be undesirable. In particular, there is a need for an improved surgical liquid jet instrument which minimizes trauma to tissue adjacent to tissue being ablated by the liquid jet. It is difficult to achieve the purposes of many surgical procedures if tissue removal cannot be confined to a desired area, or if functional surfaces adjacent to the operating site are damaged.

### SUMMARY OF THE INVENTION

Disclosed herein are a series of devices related to surgical procedures utilizing liquid jets for cutting, ablating, sculpting, trimming, etc., tissues and/or materials from the body of a patient. The invention includes, in one aspect, a series of devices comprising surgical liquid jet instruments for forming a liquid jet, in another aspect, methods for using the surgical liquid jet instruments, and, in yet another aspect, methods for forming certain components of the surgical liquid jet instruments. In certain surgical method embodiments of the invention using certain embodiments of the inventive liquid jet instrument, the cutting or ablating power of the liquid jet may be adjusted or controlled by an operator of the instrument, for example by varying the pressure of the liquid supplied to form the jet, to allow for improved tissue differentiation and to reduce inadvertent damage to surrounding tissues when cutting or ablating the target tissue. Liquid jet instruments of the invention may also be operated in certain inventive surgical procedures to avoid thermal damage to surrounding tissues that is often caused by instruments such as lasers and electrosurgical devices.

In one aspect, the invention provides a surgical liquid jet device that minimizes the trauma to the tissue surrounding the excised tissue. According to one embodiment of the present invention, the edges of the terminal tip of an evacuation tube, as well as, in certain embodiments, the edges of the terminal tip of the pressure tube at the distal end of the instrument, are “blunted,” i.e. smoothed, rounded, and/or repositioned/deflected with respect to a center axis of the lumen, at the terminal tip, formed by the tube, etc., to a sufficient extent so as to be substantially non-traumatic to tissue against which the terminal tip(s) of the tube(s) may be brought into contact in normal usage for procedures for which the instrument is indicated. Moreover, in some embodiments, the method of blunting the edges provides a desired narrowed opening of the evacuation lumen, which may be helpful in maintaining evacuation of liquid and debris from the tissue site. The term “terminal tip” as used herein in the context above, refers to either the region at the inlet end of the evacuation tube that circumscribes the jet-receiving opening of the evacuation tube or the region at the outlet end of the pressure tube that forms, defines, or circumscribes the nozzle, depending on whether this term is modifying the evacuation tube or the pressure tube, respectively. The “terminal tip” of both of the evacuation tube and the pressure tube are typically located at the “distal end” of the surgical liquid jet instrument, as that term is defined below.

In one aspect, the invention provides a surgical instrument having a distal end adapted to perform a surgical procedure on a patient and a proximal end adapted to be controllable by an operator. The instrument includes a pressure tube having a pressure lumen defined by a wall of the pressure tube, the pressure tube having sufficient burst strength to conduct a high pressure liquid towards the distal end of the instrument, and the pressure tube includes at least one nozzle providing a jet opening. The instrument also includes an evacuation tube having an evacuation lumen defined by a wall of the evacuation tube, where the evacuation lumen includes a jet-receiving opening locatable opposite the jet opening to receive a liquid jet when the instrument is in operation. The nozzle is shaped to form the liquid jet as a liquid at high pressure flows therethrough, and the evacuation tube wall has a blunted terminal tip.

In another aspect, the invention provides a surgical instrument having a distal end adapted to perform a surgical procedure on a patient and a proximal end adapted to be controllable by an operator. The instrument includes a pressure tube having a pressure lumen defined by a wall of the pressure tube, the pressure tube having sufficient burst strength to conduct a high pressure liquid towards the distal end of the instrument, and the pressure tube includes at least one nozzle providing a jet opening. The instrument also includes an evacuation tube having an evacuation lumen defined by a wall of the evacuation tube, where the evacuation tube includes a jet-receiving opening locatable opposite the jet opening to receive a liquid jet when the instrument is in operation. The nozzle is shaped to form a liquid jet as a liquid at high pressure flows therethrough. The instrument also includes a terminal tip of the evacuation tube having a center axis and a perimeter, where the terminal tip of the evacuation tube wall is curved and/or is angled inwardly towards the center axis around a majority of the perimeter of the evacuation tube.

In another aspect, the invention provides a method comprising inserting a surgical liquid-jet instrument into a surgical site in the body of a patient, creating a liquid jet with the surgical liquid-jet instrument, directing the liquid jet towards a jet-receiving opening of an evacuation tube of the surgical liquid-jet instrument, where the evacuation tube wall has a blunted terminal tip, and cutting or ablating a selected tissue within the surgical site with the liquid jet.

In yet another aspect, the invention provides a method comprising inserting a surgical liquid jet instrument into the spine of a patient, e.g. into an intervertebral disc of the patient, and cutting, ablating, and/or removing with a liquid jet of the instrument a first tissue within the spine while not cutting, ablating, and/or removing with the liquid jet of the instrument a second tissue within the spine.

In another aspect, the invention provides a method of manufacturing a surgical liquid jet instrument, the method comprising forming a blunted terminal tip on an evacuation tube wall of the surgical liquid jet instrument. The pressure tube of the instrument comprises a pressure lumen defined by a wall of the pressure tube, and the pressure tube has sufficient burst strength to conduct a high pressure liquid towards a distal end of the instrument. The pressure tube includes at least one nozzle providing a jet opening, where the nozzle is shaped to form a liquid jet as a liquid at high pressure flows therethrough. The evacuation tube comprises an evacuation lumen defined by a wall of the evacuation tube, and the evacuation tube includes a jet-receiving opening having a cross-sectional area and locatable opposite the jet opening.

The accompanying drawings are schematic and are not intended to be drawn to scale. In the figures, each identical, or substantially similar component that is illustrated in various figures is typically represented by a single numeral or notation. For purposes of clarity, not every component is labeled in every figure, nor is every component of each embodiment of the invention shown where illustration is not necessary to allow those of ordinary skill in the art to understand the invention. In the drawings:

### BRIEF DESCRIPTION OF THE DRAWINGS

FIG. 1 is a schematic illustration of a surgical liquid jet system;

FIG. 2a is a partially-cutaway schematic illustration of a portion of the distal end of a surgical liquid jet instrument for use in a surrounding liquid environment;

FIG. 2b is a partially-cutaway schematic illustration of a portion of the distal end of a surgical liquid jet instrument for use in a surrounding liquid environment, where the evacuation lumen includes a constriction;

FIG. 2c is a schematic illustration of a portion of the distal end of a surgical liquid jet instrument, illustrating various geometric relationships;

FIG. 2d is a partially-cutaway schematic illustration of a portion of the distal end of a surgical liquid jet instrument for use in a surrounding gaseous environment;

FIG. 2e is a partially-cutaway schematic illustration of a portion of the distal end of a surgical liquid jet instrument for use in a surrounding gaseous environment, where the evacuation lumen includes a constriction;

FIG. 3a is a partially-cutaway schematic illustration of a portion of a surgical liquid jet instrument, the portion including the distal end of the surgical liquid jet instrument;

FIG. 3b is a partially-cutaway schematic illustration of a portion of a surgical liquid jet instrument, the portion including the distal end of the surgical liquid jet instrument;

FIG. 3c is a partially-cutaway schematic illustration of a portion of a surgical liquid jet instrument, the portion including the distal end of the surgical liquid jet instrument;

FIG. 4a is a schematic cross-sectional illustration of a portion of one embodiment of a liquid jet surgical instrument;

FIG. 4b is a schematic perspective illustration of the portion of one embodiment of a liquid jet surgical instrument illustrated in FIG. 4a;

FIG. 5 is a schematic cross-sectional illustration of the distal portion of an evacuation tube, in which the evacuation tube has a blunted terminal tip;

FIG. 6a is a schematic cross-sectional illustration of the distal end of an evacuation tube and a cupping device for forming a blunted terminal tip of the evacuation tube in a configuration prior to blunting;

FIG. 6b is a schematic cross-sectional illustration of the distal end of an the evacuation tube and cupping device for forming a blunted terminal tip of the evacuation tube of FIG. 6a, in a configuration after blunting;

FIG. 7 is a partially-cutaway schematic illustration showing one relationship of the evacuation tube similar to the evacuation tube shown in FIG. 5 to a jet emitting pressure tube;

FIG. 8a is a schematic cross-sectional illustration of a distal end of an evacuation tube;

FIGS. 8b-8e are schematic cross-sectional illustrations of various embodiments of a blunted terminal tip of an evacuation tube according to certain embodiments of the invention;

FIGS. 9a-9d are schematic cross-sectional illustrations of additional embodiments of a blunted terminal tip of an evacuation tube according to certain embodiments of the invention; and

FIGS. 9e-9f are schematic cross-sectional illustrations showing the formation of a blunted terminal tip of an evacuation tube by the addition of an attachment, such as an external collar.

### DETAILED DESCRIPTION

The present invention provides a variety of liquid jet instruments useful in a variety of applications, many of which are especially well suited for a variety of surgical procedures. The liquid jet instruments provided by the invention can be configured in a variety of different ways for use in various surgical operating fields. Certain surgical instruments, according to the invention, are configured as surgical handpieces having a proximal end with a grasping region, or handle, shaped and configured to be comfortably held in the hand of an operator. The instruments also have a distal end that includes at least one nozzle for forming a liquid jet. The distal end of the inventive surgical instruments is used to perform a surgical procedure on a patient. Although the liquid jet instruments described herein are shown as having a handpiece configuration, it should be understood that the invention is not strictly limited to surgical handpieces, and that the invention may also be practiced utilizing liquid jet instruments having a variety of configurations and purposes. The liquid jet instruments provided by the invention can be used in a wide variety of surgical applications to utilize a high pressure liquid stream to cut, drill, bore, perforate, strip, delaminate, liquefy, ablate, shape, or form various tissues, organs, etc. of the body of a patient.

Certain embodiments of the liquid jet surgical instruments provided by the invention include a pressure tube, having a terminal end defining, forming, or circumscribing in at least one nozzle providing a liquid jet opening, and having a proximal end that is connectable to a source of liquid under high pressure, supplied, for example, by a high pressure pump or liquid dispenser. The liquid jet nozzle is shaped to form a liquid jet as a liquid under high pressure flows through the nozzle, as described below. The liquid jet, in certain embodiments, can be used to cut, ablate, sculpt, trim, form, debride, etc., various tissues of a patient in surgical procedures. In certain embodiments, the liquid pressure supplied to the instrument by the pump or dispenser is variably controllable by an operator of the instrument so that the cutting or ablating power of the liquid jet is adjustable by the operator. This adjustability of the pressure can allow an operator to create a liquid jet with the instrument that can differentiate between different types of tissue within a surgical operating field. For example, a lower pressure can be utilized for cutting or ablating a soft tissue such as fat or the nucleus pulposus of an intervertebral disc from a surface of a harder tissue, such as muscle, bone, cartilage, or the annulus fibrosus of an intervertebral disc, where the liquid jet has sufficient strength to cut or ablate the soft tissue without damaging the underlying, surrounding, adjacent, and/or interdigitated harder tissue. A higher pressure can then be selected that is sufficient to form a liquid jet capable of cutting or ablating hard tissue, such as muscle or bone. In this way, a liquid jet surgical instrument provided by certain embodiments of the invention can provide highly selective and controllable tissue cutting in various surgical procedures, such as, for example, surgical procedures on the spine.

In some embodiments, an external source of suction, for example a vacuum pump or aspirator, can be provided in fluid communication with a proximal end of an evacuation lumen of an evacuation tube of the instrument in order to provide the suction driving force required for evacuating material from the surgical field via a jet-receiving opening of the evacuation tube. In certain embodiments, however, the invention provides surgical instruments having an evacuation tube that is shaped and positionable relative to the jet nozzle to enable evacuation of essentially all of the liquid comprising the liquid jet as well as ablated tissue and debris from the surgical site without requiring an external source of suction. In certain embodiments, the evacuating force created by the liquid jet being directed into the evacuation lumen is sufficient to evacuate material from the operating site to a drainage reservoir located at the proximal end of the evacuation tube or an evacuation conduit connected to the proximal end of the evacuation tube. In such embodiments, the liquid jet and the evacuation tube together can act as an eductor pump, which utilizes the momentum and kinetic energy of the moving fluid of the liquid jet to create an evacuating force capable of driving the liquid, ablated material, and debris through the evacuation lumen and away from the surgical site.

The inventive surgical liquid jet instruments, in certain embodiments, can be configured to effectively remove material from a surgical site and transport the material through an evacuation lumen without the need for an external source of suction, for a wide variety of angular orientations between the central region of the liquid jet and the longitudinal axis of the evacuation lumen. The term “central region of the liquid jet” as used herein refers to a region defining the geometric center of the liquid jet. This region is typically an essentially cylindrical region of the liquid jet confined within a cylinder whose outer surface has a shape and perimeter defined by the inner circumference of the liquid jet opening, which circumference is projected from the liquid jet opening to the jet-receiving opening along an axis that is co-linear with the longitudinal axis of the jet nozzle. The “longitudinal axis” of the jet nozzle, as will be described in more detail below, is defined by the axial center line of the nozzle region of the pressure tube, which is typically at the terminal tip of the pressure tube. The “longitudinal axis” of the evacuation lumen refers to an axis defining the geometric center of the evacuation lumen in a region that is proximal to the jet-receiving opening. In typical embodiments, this region of the evacuation lumen will have a longitudinal axis that is essentially parallel to the longitudinal axis of the elongated body of the instrument, which is held and controlled by the hand of the operator. As used herein in the context of describing geometric relationships between longitudinal axes of various components, the term “co-linear” refers to components whose longitudinal axes are superimposed on essentially the same line in space. The term “parallel” when used in the same context herein refers to longitudinal axes that are not co-linear, but that are oriented in an essentially identical direction in space. Accordingly, the surgical instruments provided by the invention, in certain embodiments, can enable effective evacuation of material and debris from the surgical site, without the need for an external source of vacuum, for a wide variety of liquid jet angular configurations, including instruments providing liquid jets that are directed axially, transversely, or at any angle between 0 and 180° with respect to a longitudinal axis defining the proximal end, or body, of the surgical instrument.

Such flexibility allows certain embodiments of the inventive surgical instruments to be designed having a distal end that has a variety of predetermined contours, shapes, and sizes specifically selected for particular surgical procedures. Such customization of the instruments can allow certain embodiments of the liquid jet instruments to be designed and configured to facilitate and reduce the difficulty of insertion of the distal end of the device into confined regions of the body defining a surgical operating space. For example, as will be discussed in greater detail below, the invention provides surgical liquid jet instruments and a surgical method for performing surgical procedures on the spine of a patient.

Certain embodiments of the inventive liquid jet surgical instruments may include distal ends that are designed and configured to prevent or reduce plugging of the evacuation lumen, blow-by of the liquid jet, or back spray or misting of the liquid jet when the instrument is in operation. “Blow-by” of the liquid jet, as used herein, refers to a portion of the liquid jet, or a high velocity fluid entrained by the liquid jet (comprising the “entrainment region” as discussed below), having a cross-sectional area, at the plane of the jet-receiving opening, that is larger than the cross-sectional area of the jet-receiving opening so that at least a portion of the liquid jet or high velocity fluid misses or “blows by” the jet-receiving opening. Blow-by is generally undesirable because it can lead to unintended tissue damage and poor evacuation efficiency. “Back spray” as used herein refers to a liquid jet, or high velocity fluid entrained by the liquid jet, entering the jet-receiving opening in the evacuation tube and subsequently reflecting or flowing back into the surgical field from the jet-receiving opening. Such back spray is undesirable in operation due to the potential of contamination of the surgical operating field and/or aerosolization of infective material, in addition, back spray typically indicates a poor efficiency level of the evacuation of material by the instrument via eductor pump action. As described in more detail below, the surgical instruments provided by the invention, in certain embodiments, substantially reduce, and in certain embodiments essentially eliminate, performance problems associated with blow-by and back spray when the instruments are in operation.

Plugging of the evacuation lumen can be prevented, for certain embodiments involving surgical instruments designed for operation in a liquid environment, by constructing the evacuation tube to have a region that is within and/or downstream of the jet-receiving opening that is designed to be able to macerate at least a portion of the tissue entrained by the liquid jet into a plurality of particles when the instrument is in operation. The term “macerate” as used herein refers to a disaggregation of entrained material, for example an entrained tissue, by a liquid within the evacuation lumen undergoing intensely turbulent flow that creates a region of extremely high fluid shear and impacting forces capable of partitioning the material into particles having a size small enough to pass through the evacuation lumen without plugging the lumen. In certain embodiments, the evacuation tube is able to macerate a substantial fraction of the tissue entrained into a plurality of essentially microscopic particles. “Microscopic” as used herein refers to particles having a dimension too small to be visualized unaided by the human eye.

Prevention of blow-by and back spray can be accomplished by providing a surgical liquid jet instrument having a distal end configured so that when in operation, the liquid jet and the high velocity fluid entrained by the liquid jet occupies a substantial fraction of the cross-sectional area of the jet-receiving opening, but does not occupy a region larger than the cross-sectional area of the jet-receiving opening. As discussed in more detail below, this “substantial fraction” refers to at least 50%, but less than 100% of the cross-sectional area of the jet-receiving opening being occupied by an entrainment region created by the liquid jet.

The inventive surgical liquid jet instruments will now be described in more complete detail in the context of several specific embodiments illustrated in the appended figures. It is to be understood that the embodiments described are for illustrative purposes only and that the novel features of the invention, as described in the appended claims, can be practiced in other ways or utilized for instruments having other configurations, as apparent to those of ordinary skill in the art.

At the outset, it should be noted that a detailed treatment and discussion of a wide variety of design parameters, configurations, materials of construction, and other aspects of the design, fabrication, and construction of liquid jet surgical instruments are provided in commonly owned U.S. Pat. Nos. 5,944,686; 6,375,635; and 6,511,493; in U.S. Patent Application Publication Nos. 2003/0125660 A1, 2004/0243157 A1, and in International Application No. 2004/069064 A2, each of which is incorporated herein by reference. The reader is referred to these issued patents and patent publications for detailed description of and guidance as to the construction and design of certain embodiments of the liquid jet components of the instruments described herein. For example, U.S. Pat. No. 6,375,635 describes in detail design considerations related to the configuration and sizing of the nozzle, evacuation lumen, liquid jet length and dispersion, materials of construction, liquid pressures for operation, etc. for liquid jets configured to directly contact, cut and/or fragment and/or disaggregate tissue and facilitate removal of tissue through an evacuation lumen. Accordingly, while certain specific design parameters are called out and discussed in more detail below, others that may not specifically mentioned or discussed are discussed in detail in one or more of the above-referenced U.S. Patents or Patent Publications. Such parameters, configurations and design considerations disclosed in these references can be, in many cases, applicable to and useful for practicing many aspects of the current invention.

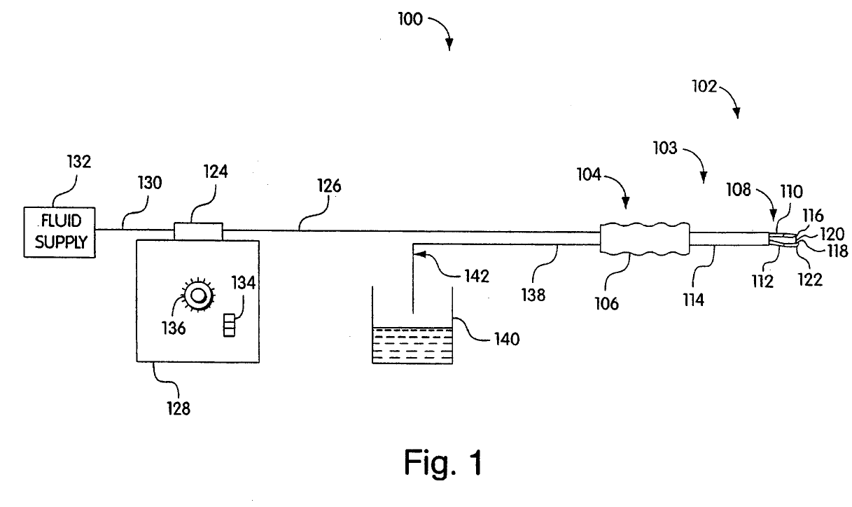

FIG. 1 shows one embodiment of a liquid jet surgical system 100 utilizing a liquid jet surgical instrument 102, according to an embodiment of the invention. The surgical instrument 102 is configured as a surgical handpiece having a proximal end 103 including a body 104 having a grasping region 106 configured for placement in the hand of an operator of the instrument. The surgical instrument 102 has a distal end 108 including a tube 110 forming a pressure lumen and a tube 112 forming an evacuation lumen. “Distal end” when used herein in the context of a region of a surgical instrument refers to the portion of the surgical instrument that is adapted to perform a surgical procedure on a patient, and which is inserted into a surgical site during operation of the instrument. The distal end 108 of the instrument 102 may, in some embodiments, comprise only the distal ends of pressure tube 110 and evacuation tube 112, or in other embodiments, may include components proximal to the distal ends of the pressure tube 110 and the evacuation tube 112 that are also inserted into a surgical operating space of the patient during use of the instrument. In the illustrated embodiment, surgical instrument 102 further includes a sheath 114, which at least partially surrounds pressure tube 110 and evacuation tube 112 and supplies support for the tubes to assist in maintaining and/or establishing a desired geometric configuration between the pressure tube and the evacuation tube, when the instrument 102 is in operation. The pressure lumen formed by tube 110 further includes at the terminal tip at its distal end a nozzle 116, which forms a liquid jet as a high pressure liquid supplied by pressure tube 110 streams therethrough. The evacuation lumen formed by tube 112 includes a jet-receiving opening 118 located at the terminal tip at its distal end and positioned, when the instrument 102 is in operation, opposite the jet nozzle 116 at a predetermined distance therefrom in order to receive the liquid jet 120. In the particular embodiment illustrated, liquid jet 120 is directed transversely (e.g., at an angle of approximately 90°) with respect to the longitudinal axes of the evacuation lumen and the body 104 of the instrument 102. As will be explained in more detail below, for such embodiments, the evacuation lumen wall 112 may include a jet-deflecting portion 122 downstream and adjacent to the jet-receiving opening 118 that is utilized to deflect and direct the liquid entering the jet-receiving opening 118 proximally within the evacuation lumen.

Pressure tube 110 and evacuation tube 112 are preferably constructed from a surgical grade stainless steel, however, in alternative embodiments, either or both of the tubes may be constructed from other suitable materials, for example certain polymeric materials, as apparent to those of ordinary skill in the art. Regardless of the specific material from which the pressure tube is constructed, the pressure tube must have sufficient burst strength to enable it to conduct a high pressure liquid to nozzle 116 to form liquid jet 120. The burst strength of the pressure tube should be selected to meet or exceed the highest contemplated pressure of the liquid supplied for use in the specific surgical procedure to be performed. Typically, surgical instrument 102 will operate at liquid pressure between about 500 psig and about 50,000 psig, depending on the intended material to be cut and/or ablated. Those of ordinary skill in the art will readily be able to select appropriate materials for forming pressure tube 110 and evacuation tube 112 for particular surgical requirements.

In certain embodiments, pressure tube 110 and evacuation tube 112 are constructed and supported so that the distal ends of the walls of the tubes are sufficiently stiff to prevent deflection of the tubes by, for example, contact of the walls with surfaces within the surgical operating space, which deflection could potentially lead to misdirection of liquid jet 120 so that it is no longer incident upon jet-receiving opening 118, thus potentially causing unintended tissue damage to the patient.

Pressure tube 110 is in fluid communication with high pressure pump 124 via high pressure liquid supply conduit 126. High pressure liquid supply conduit 126 also has a burst strength capable of withstanding the highest liquid pressures contemplated for using the instrument 102 for a particular surgical application. In some embodiments, high pressure liquid supply conduit 126 comprises a burst-resistant stainless steel hypotube constructed to withstand at least 50,000 psig. In some embodiments, the hypotube may be helically coiled to improve the flexibility and maneuverability of the surgical instrument 102. In certain embodiments, high pressure liquid supply conduit 126 comprises a Kevlar reinforced nylon tube that is connectable to the pressure tube 110.

In fluid communication with high pressure liquid supply conduit 126 is a high pressure pump 124, which can be any suitable pump capable of supplying the liquid pressures required for performing the desired surgical procedure. Those of ordinary skill in the art will readily appreciate that many types of high pressure pumps may be utilized for the present purpose, including, but not limited to, piston pumps and diaphragm pumps. In certain embodiments, high pressure pump 124 comprises a disposable piston or diaphragm pump, which is coupled to a reusable pump drive console 128. High pressure pump 124 has an inlet that is in fluid communication with a low pressure liquid supply line 130, which receives liquid from liquid supply reservoir 132. Pump drive console 128 may include an electric motor that can be utilized to provide a driving force to high pressure pump 124 for supplying a high pressure liquid in liquid supply conduit 126.

While a variety of known pump consoles may be utilized in the context of the present invention, certain pump drive consoles include a constant speed electric motor that can be turned on and off by means of an operator-controlled switch 134. In certain embodiments, operator-controlled switch 134 comprises a foot pedal or a button or trigger located on grasping region 106 of the surgical instrument 102 that may be easily accessed by the operator of the instrument. In some embodiments, pump drive console 128 can have a delivery pressure/flow rate that is factory preset and not adjustable in use. In other embodiments, the pressure/flow rate may be controlled by the operator via an adjustable pressure/flow rate control component 136, that can control the motor speed of the pump drive console and/or the displacement of the high pressure pump. While in FIG. 1, pressure/flow rate control component 136 is illustrated as a knob on pump drive console 128, in certain embodiments, such component would comprise a foot pedal, or trigger/button located on grasping region 106, as previously discussed for on/off control of the pump drive console 128. In yet other embodiments, pump drive console 128 and high pressure pump 124 may be replaced by a high pressure liquid dispenser or other means to deliver a high pressure liquid, as apparent to those of ordinary skill in the art. In certain embodiments, a pumping system such as one of those described in commonly-owned U.S. Patent Application Publication Nos. 2002/0176788 or 2004/0228736, both incorporated herein by reference, could be used.

The liquid utilized for forming the liquid cutting jet can be any fluid that can be maintained in a liquid state at the pressures and temperatures contemplated for performing the surgical procedures. For applications in which the instruments are used to perform surgical procedures in a live patient, the liquid utilized should also be physiologically compatible. In typical embodiments, the liquid supplied will be a sterile surgical saline solution, or sterile water and liquid supply reservoir 132 can comprise a sterile container, such as an intravenous (IV) bag containing such fluid. In some embodiments, in order to improve the cutting or ablating character of the liquid jet, the liquid may contain solid abrasives, or the liquid may comprise a liquefied gas, for example carbon dioxide, which forms solid particulate material upon being admitted from nozzle 116 to for the liquid jet 120. In other embodiments, the liquid supplied to surgical instrument 102 may include medicaments, such as antiseptics, antibiotics, antiviral components, anesthetics, drugs, chemotherapy agents, etc., that are useful in the context of a specific surgical procedure. In other embodiments, the fluid may include a dye to improve visualization of the liquid jet when the instrument is in operation.

Evacuation tube 112 is connectable at its proximal end to an evacuation conduit 138, which can be used to transport evacuated material and debris to a drainage reservoir 140. The liquid contained in evacuation conduit 138 is under relatively low pressure and, accordingly, evacuation conduit 138 may be constructed, in certain embodiments, of a low cost flexible material, for example, polymeric tubing, such as polyvinyl chloride (PVC), silicone, polyethylene, rubber, etc. tubing. In certain embodiments, evacuation conduit 138 should have a minimum internal cross-sectional area that equals or exceeds the maximum internal cross-sectional area of the evacuation lumen. In the illustrated embodiment, surgical instrument 102 is constructed such that the evacuation lumen is capable of evacuating liquid jet 120 and ablated material and debris from the jet-receiving opening 118 to the proximal end of the evacuation lumen and through evacuation conduit 138 into drainage reservoir 140, without the need for an external source of suction. In such embodiments, evacuation conduit 138 may include a vacuum breaker 142 or a proximal end that is not couplable to an external source of suction, so that it is not possible for an operator to inadvertently couple evacuation conduit 138 to an external source of suction when the instrument is in operation.

In certain embodiments, the fluid supply path of liquid jet surgical system 100 is disposable, and sterilizable, for example by chemical methods such as exposure to ethylene oxide, or by gamma or beta irradiation, as apparent to those of ordinary skill in the art. In certain embodiments, the fluid path is supplied pre-sterilized to the user for a single use only. Those of ordinary skill in the art understand what is meant by “disposable” and “for a single use only.” Disposability of the liquid supply path, including liquid supply reservoir 132, liquid supply line 130, high pressure pump 124, high pressure liquid supply conduit 126, and pressure tube 110 is advantageous because such components can be difficult to effectively clean and sterilize between use without reducing the utility of the instrument, for example by the plugging of jet nozzle 116 with deposits during the sterilization process. In certain embodiments, all of the components of liquid jet surgical system 100 are entirely disposable after a single use except for pump drive console 128. For embodiments where the surgical liquid jet instrument is disposable after a single use, the instrument may be sterilizable, and may be provided pre-sterilized. In other embodiments, only the pressure tube and the distal end of the instrument for insertion into the patient are sterilizable or pre-sterilized.

The present invention provides, in certain embodiments, surgical liquid jet instruments which are specifically designed and constructed for use in a particular surgical environment. Specifically, in some embodiments, the present invention provides surgical liquid jet instrument designs that are tailored to provide highly desirable performance characteristics in surgical operating environments where the liquid jet is submerged in a liquid environment when the instrument is in operation, and, in other embodiments, the present invention provides surgical liquid jet instrument designs that are tailored to provide highly desirable performance characteristics in surgical operating environments where the liquid jet is surrounded by a gaseous environment when the instrument is in operation. More specifically, the invention provides surgical liquid jet instruments including pressure tubes and evacuation tubes that are shaped, and positioned relative to each other, to establish certain predetermined geometric relationships between the jet forming components and jet-receiving components that are specifically selected to provide the desired performance characteristics of the instrument in a liquid or gaseous surgical environment. Importantly, the above mentioned geometric relationships and design characteristics may be substantially different for instruments that are designed for use in a liquid environment when compared to instruments that are designed for use in a gaseous environment.

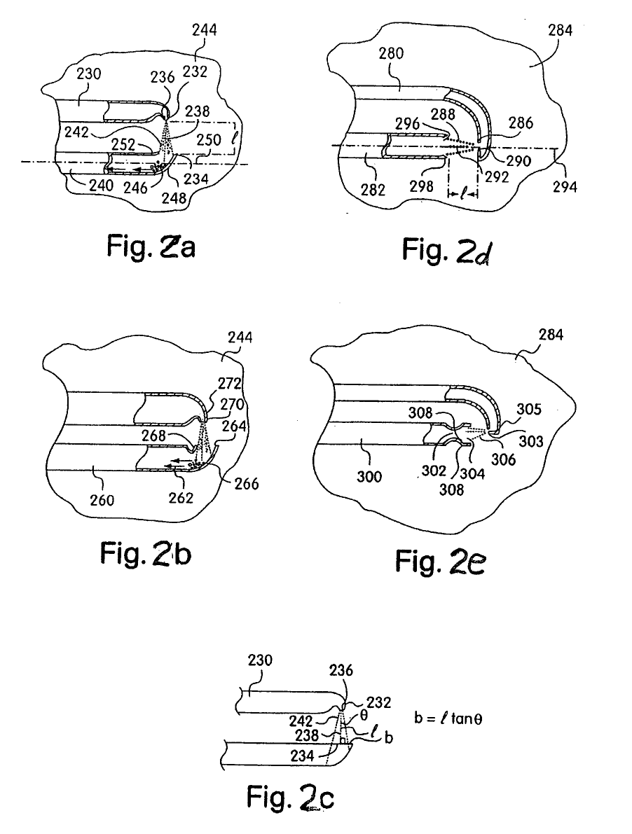

Reference is made to FIG. 2 for describing the operation and design characteristics of certain devices for use in forming a liquid jet, and in particular one that is submerged in a surrounding liquid-containing surgical environment. FIG. 2a shows a partially cutaway view of the distal ends of pressure tube 230 and evacuation tube 240, which can form part of a surgical instrument, for example such as that shown previously in FIG. 1. Prior to operation, the distal ends of pressure tube 230 and evacuation tube 240 would be inserted into the operating field and at least partially submersed in a liquid 244 therein so that at least nozzle 232 and jet-receiving opening 234 are completely surrounded by liquid 244. When the instrument is in operation, liquid under high pressure is delivered via the pressure lumen to nozzle 232, causing jet opening 236 to create a liquid jet 238 as the high pressure liquid streams therethrough. As mentioned previously, for embodiments where the liquid jet 238 is formed in a surrounding liquid environment 244, jet 238 may be substantially collimated as it exits jet opening 236. The more collimated a liquid jet, the less the liquid jet will diverge or disperse as it traverses the gap between jet opening 236 and jet-receiving opening 234. Thus, a highly collimated jet will have a cross-sectional shape and area at the jet-receiving opening 234 that is substantially similar to the cross-sectional shape and area of the liquid jet at jet opening 236.

As discussed previously, the pressure of the high pressure liquid supplied to nozzle 232 for forming the liquid jet 238 depends on the particular design of nozzle 232 and the hardness/toughness of tissue or material to be cut or ablated. In certain embodiments, the liquid at high pressure is supplied to jet opening 236 at a pressure of at least 500 psig, in other embodiments at a pressure of at least about 1,000 psig, 2,000 psig, 3,000 psig, or 5,000 psig, and still other embodiments at a pressure of at least about 10,000 psig, or 15,000 psig, and still other embodiments at a pressure of at least 20,000 psig, and in yet still other embodiments at a pressure of at least about 30,000 psig, or 50,000 psig. Also as discussed previously, for embodiments where a collimated jet is desired, nozzle 232 may have a length to minimum internal diameter ratio of at least about four, about six, and in other embodiments at least about ten. Using other nozzle-forming technology, such as that described in commonly-owned International Application Publication No. WO 2004/069064, different ratios or design criteria may apply. Jet opening 236 can have a circular cross-sectional area, but may, in other embodiments, have other cross-sectional shapes, such as rectangular, oval, slit-like, etc., for forming jets having different shapes for specific desired purposes. In certain embodiments, jet opening 236 has an internal diameter of between about 0.001 and about 0.02 inches, in certain embodiments between about 0.003 and about 0.01 inches, and in certain embodiments about 0.005 inches.

Liquid jet 238, which is collimated as it exits jet opening 236, tends to create a visible, opaque entrainment region 242 surrounding liquid jet 238. Entrainment region 242 is comprised of rapidly moving liquid, which is entrained and driven by the kinetic energy of liquid jet 238. Liquid jet 238, as it rapidly moves through liquid environment 244, also tends to create a zone of low pressure, which is essentially coextensive with entrainment region 242. In certain embodiments involving high pressure liquids and rapidly moving liquid jets, the pressure in entrainment region/low pressure zone 242 will be lower than the vapor pressure of the surrounding liquid in liquid environment 244, thus causing cavitation of the liquid in entrainment region 242 and a resulting formation of an abundance of extremely small gas bubbles 246 within the liquid in the entrainment region 242, making the region visually opaque.

As discussed previously, it is desired, in certain embodiments, for safety and performance, that the instrument be designed to reduce, and preferably eliminate, undesirable effects, such as blow-by of the liquid jet, plugging of the jet-receiving opening of the evacuation tube, and inefficient tissue/debris entrainment and removal. Also, as previously mentioned, in certain embodiments, it is desirable that ablated tissue and debris be evacuated from the surgical site through the evacuation lumen, without the need for a source of external suction to be applied to the proximal end of the evacuation lumen. In order to provide the above-mentioned characteristics, the inventive surgical instruments for use in a liquid environment can include an evacuation tube having a specifically selected predetermined shape and configuration, which is positionable relative to the jet opening at a specific predetermined distance. Specifically, in certain embodiments, jet-receiving opening 234 is positioned, when the instrument is in operation, opposite jet opening 236, at a distance l therefrom, and the instrument is provided with a nozzle 232 having a length to minimum diameter ratio so that essentially all of the fluid in liquid jet 238 enters jet-receiving opening 234. As discussed above, liquid jet 238 will tend to create entrainment region 242 surrounding the liquid jet 238 when the instrument is in operation. Entrainment region 242 will typically be symmetrically disposed around liquid jet 238 and will tend to diverge in a direction from jet opening 236 to jet-receiving opening 234. In embodiments where jet opening 236 is circular in shape, entrainment region 242 will have a truncated cone shape, having a truncated apex at jet opening 236 and a base defined as a cross section of the cone at the plane of jet-receiving opening 234. In certain embodiments, the base of entrainment region 242 occupies between about 50% and about 100% of the cross-sectional area of jet-receiving opening 234 when the instrument is in operation; in certain embodiments the entrainment region occupies at least about 75%; in certain embodiments at least about 90%; in certain embodiments at least about 95% of the cross-sectional area of jet-receiving opening 234 when the instrument is in operation.

As shown in FIG. 2c, the cross-sectional area of the jet-receiving opening 234 required to ensure that the entrainment region 242 occupies the desired relative fraction of the cross-sectional area of the jet-receiving opening 234, as discussed above, is functionally related to the chosen distance l between the jet opening 232 and the jet-receiving opening 234 and the degree of divergence characterizing the entrainment zone (represented by angle θ in FIG. 2c). Specifically, the desired cross-sectional radius b of the base of the entrainment region 242 at the jet-receiving opening 234 is related to distance l and the degree of divergence of the entrainment region by b=l tan θ. Distance l is typically selected based on the desired use of the surgical instrument, dictating a required fluid path cutting/ablating length. Based upon this desired distance l, the required size of the jet-receiving opening 234 may be determined experimentally by submersing the pressure tube 230 and nozzle 232 in a liquid environment 244, forming a liquid jet 238 by supplying a liquid to the nozzle 232 at a desired pressure, and visually observing the size of the entrainment region 242 or cavitation cone created around the liquid jet 238, and estimating angle θ from the observations.

As mentioned above, the separation distance l between the jet opening 236 and the jet-receiving opening 234 depends upon the requirements of the particular surgical procedure for which the surgical instrument is used; however, for some typical embodiments, the distance will have a maximum value of about 1 cm, for other typical embodiments, about 5 mm, and for yet other typical embodiments, about 1 mm. The jet-receiving opening 234 may have a diameter of between about 0.01 and about 0.2 inches, in other embodiments between about 0.03 and about 0.1 inches, and in some embodiments a diameter of about 0.06 inches.

Referring again to FIG. 2a, a detailed configuration for evacuation tube 240 will now be described. Certain embodiments of evacuation tube 240 for use in surgical instruments intended to be operated in a liquid environment include a maceration region 246 within and/or downstream and in close proximity to the inlet to evacuation tube 240 at jet-receiving opening 234. Maceration region 246 is defined as a region that contains a liquid undergoing intensely turbulent flow and impacting an internal surface of the wall of the evacuation tube at an acute angle, thus creating significant impacting forces capable of macerating entrained material/tissue, when the instrument is in operation. The combination of the intensely turbulent flow of the liquid in maceration region 246 and the impacting forces of liquid jet 238 and the liquid in entrainment region 242 against the wall of the evacuation tube enable the liquid within the maceration region to macerate at least a portion of any tissue or material entrained by the liquid in entrainment region 242 into a plurality of small particles. In certain embodiments, the maceration region is able to macerate a substantial fraction (i.e., the majority of) the entrained tissue into a plurality of small particles. In certain embodiments, the plurality of particles at least partially comprises a plurality of microscopic particles too small to be seen unaided with the human eye. The particles should be small enough to pass through evacuation lumen without plugging the evacuation lumen, when the instrument is in operation.

In order to provide a maceration region, evacuation tube 240 may include a jet-deflecting portion 248 that is located adjacent to and downstream of jet-receiving opening 234. Jet-deflecting region 248 may be either a straight surface that is angled with respect to the direction of at least a central portion of liquid jet 238, or in certain embodiments, jet-deflecting region 248 comprises a smoothly curved surface upon which at least a portion of liquid jet 238 impinges, where the curved surface is shaped to deflect at least a portion, and in certain cases, all of the liquid jet 238 and liquid comprising entrainment region 242 in a direction that is essentially parallel to the longitudinal axis 250 of the lumen of evacuation tube 240 in the region proximal to the jet-deflecting region 248. In certain embodiments, the radius of curvature of the curved surface defining jet-deflecting region 248 is essentially constant, having a value of between about 1 and 20 times the internal diameter of evacuation tube 240. It is also a feature of certain embodiments of the surgical instruments provided by the invention that the liquid jet be directed into the jet-receiving opening so that a direction of at least a central portion of the liquid jet forms an angle of no greater than 10 degrees with respect to a line normal (i.e., perpendicular) to a plane defining (i.e., co-planar to) the jet-receiving opening. In certain embodiments, the central portion of the liquid jet is essentially parallel to a line that is normal to the plane defining the jet-receiving opening.

To provide effective eductor pump action of the evacuation tube, in some embodiments, the lumen of evacuation tube 240 will have an essentially constant internal cross-sectional area from jet-receiving opening 234 to a position that is proximal to the distal end of the surgical instrument where the proximal end of the evacuation lumen is located. In other embodiments, eductor pump action can be enhanced by providing an evacuation lumen having an essentially constant cross-sectional area and having a jet-receiving opening, which has a cross-sectional area that is less than the cross-sectional area of the evacuation lumen (i.e., the internal cross-sectional area of the evacuation lumen has a minimum value at the jet-receiving opening). In yet other embodiments, eductor pump action can be enhanced by providing an evacuation lumen having an internal cross-sectional area which increases continuously from a minimum value at the jet-receiving opening to a maximum value at a position located proximal to the jet-receiving opening. In such embodiments, this maximum value of the internal cross-sectional area may be essentially constant for positions within the evacuation lumen that are proximal to the above-mentioned position. In each of the above-mentioned embodiments, there may be essentially no reductions in the internal cross-sectional area of the evacuation lumen at any position proximal and/or downstream of the maceration region described above.

FIG. 2b shows an alternative design embodiment for the construction of the evacuation tube for surgical instruments designed for use in a liquid surgical environment. Evacuation tube 260 includes a constriction 262 which creates a reduction in the internal cross-sectional area of the evacuation lumen. The constriction 262 is located proximal to jet-receiving opening 264, and may be positioned immediately proximal and adjacent to maceration region 266. In operation, the constriction 262 in the evacuation tube 260 will act as a venturi as liquid within the evacuation lumen flows through the constriction, thus enhancing the eductor pump action of evacuation tube. In the illustrated embodiment, constriction 262 comprises a pinch 268 in the sidewall of the tubing conduit comprising evacuation tube 260. In certain embodiments, the cross-sectional area of constriction 262 should be between about three and about eight times the cross-sectional area of jet-opening 270 in nozzle 272.

Referring again to FIG. 2a, evacuation tube 240 is shaped and positioned relative to pressure tube 230 so that at least a central portion of liquid jet 238 is directed into jet-receiving opening 234 in a direction forming a non-zero angle with respect to (i.e. non-parallel with) the longitudinal axis 250 of the lumen of evacuation tube 240 in a region proximal to jet-deflecting region 248. In some embodiments, this angle can be between about 45 and 115 degrees, in other embodiments between about 80 and 100 degrees, and in some embodiments, as illustrated, the angle may be about 90 degrees. In other embodiments, involving surgical instruments designed for use in a liquid environment, the direction of at least the central portion of the liquid jet and longitudinal axis of the lumen of the evacuation tube in a region proximal to the jet-deflecting region may be essentially parallel.

FIGS. 2d and 2e illustrate certain arrangements for liquid jet surgical instruments designed for use in a surrounding gaseous environment. Referring to FIG. 2d, a partially cutaway view of the distal ends of a pressure tube 280 and evacuation tube 282 of a surgical liquid jet instrument for use in gaseous environment 284 is shown. For instruments designed for use in a gaseous environment, nozzle 286 may have a lower length to minimum internal diameter ratio than that for nozzles employed for instruments designed for use in a liquid environment. Nozzle 286, constructed in the illustrated embodiment as a hole bored in a sidewall of pressure tube 280, may have a length to minimum internal diameter ratio not greater than about four, and in certain embodiments not greater than about two. Unlike the relatively collimated liquid jets suitable for instruments for use in a liquid environment, instruments for use in a gaseous environment may advantageously create a diverging liquid jet as a high pressure liquid flows through the nozzle. Liquid jet 288 emitted from jet opening 290, when the instrument is in operation, may be a diverging jet creating an entrainment region comprised of a diverging zone of liquid droplets 292 moving through gaseous environment 284. The zone of liquid droplets comprising liquid jet 288 will tend to create a region of relatively low gas pressure, when compared to the pressure in the gas surrounding the liquid jet region 288, that will have a tendency to entrain and draw tissue and material into an entrainment region that is essentially co-extensive with jet region 288.

Of concern for certain applications employing liquid jet surgical instruments in a gaseous environment is minimizing, and in certain cases eliminating, misting and back spray of the liquid jet from the jet-receiving opening. Such misting or back spray can cause poor visualization of the surgical field, in addition to potentially creating infectious and/or undesirable aerosolization of material into the surrounding gaseous environment. In order to avoid back spray or misting from the evacuation lumen, evacuation tube 282, in certain embodiments, is essentially straight at its distal end so that an axis 294 defining the direction of at least a central region of liquid jet 288 is essentially co-linear with the longitudinal axis of the distal end of the evacuation lumen.

As mentioned above, liquid jet 288 is in certain embodiments, a diverging jet, which diverges as it travels from jet opening 290 to jet-receiving opening 296. Diverging jet 288 may have an apex at jet opening 290 and, for essentially circular jet opening shapes, may have a truncated cone shape, where the truncated apex of the cone is located at jet opening 290 and the base of the cone is defined as the planar cross section of the cone at jet-receiving opening 296. As was previously the case for surgical instruments designed for use in a liquid environment, certain embodiments of surgical instruments designed for use in a gaseous environment provide an evacuation tube shaped and positioned relative to the jet opening, when the instrument is in operation, so that the base of the liquid jet entrainment region occupies between about 50% and about 100% of the cross-sectional area of the jet-receiving opening; in certain embodiments the entrainment region occupies at least about 75%; in certain embodiments at least about 90%, and in certain embodiments at least about 95% of the cross-sectional area of the jet-receiving opening. As was previously described in the context of instruments for use in a liquid environment, the size of jet-receiving opening 296 can be selected based upon the desired separation distance between jet opening 290 and jet-receiving opening 296 and the length to minimum internal diameter ratio of nozzle 286, which may dictate the degree of divergence of liquid jet 288. Analogous to the geometrical relationships discussed previously in the context of FIG. 2c, the relationship between the radius of liquid jet entrainment region 288 at jet-receiving opening 296 is related to separation distance l between jet opening 290 and jet-receiving opening 296 as b=l tan θ, where θ defines the divergence angle of liquid jet 288, which may be related to the length to minimum diameter ratio of nozzle 286, and b is defined as the radius of the base of the liquid jet entrainment region 288. As described before, the desired size of jet-receiving opening 296 may be determined experimentally, for example by creating a liquid jet in a gaseous environment using a desired liquid supply pressure and a given nozzle configuration, visually observing the diverging liquid jet formed, and estimating angle θ from the observation. The appropriate jet-receiving opening size can then be selected based on θ and the desired separation distance l.

In some embodiments, it is also desirable to shape and position evacuation tube 282 with respect to jet opening 290 so that the cross-sectional shape and area of liquid jet 288 at a given location 298 within the evacuation lumen is essentially the same as the internal cross-sectional shape and area of the evacuation lumen at given location 298. Given location 298 may coincide with jet-receiving opening 296 or may be located proximal to jet-receiving opening 296. For embodiments where given location 298 is located proximal to jet-receiving opening 296, the given location 298 may be selected to be no greater than about 5 mm proximal to jet-receiving opening 296. By shaping and positioning evacuation tube 282 in this fashion, liquid jet 288 can completely fill the cross-sectional area of the evacuation lumen at a position at or near its jet-receiving opening, thus essentially eliminating back spray and misting and improving evacuation via eductor pump action.

Evacuation tube 282 may have an essentially constant internal cross-sectional area, may have an essentially constant cross-sectional area with a jet-receiving opening having a cross-sectional area that is less than the cross-sectional area of evacuation tube 282, or may have an internal cross-sectional area which increases continuously from a minimum value at jet-receiving opening 296 to a maximum value at a position proximal to jet-receiving opening 296, which then remains essentially constant for positions proximal to this position. Alternatively, as shown in FIG. 2e, the instrument may include an evacuation tube 300 having a constriction 302 located proximal to a jet-receiving opening 304, where the constriction acts as a venturi to enhance the eductor pump action of the evacuation tube. For embodiments including a constriction in the evacuation tube, it may be preferred that the liquid jet 306 is directed to contact the inner surface of the wall of the evacuation tube at a given location 308 that is located distal to constriction 302. The cross-sectional area of constriction 302 may be between about three and fifteen times greater than the cross-sectional area of jet opening 303 in nozzle 305.

It should be understood that while certain embodiments of the inventive liquid-jet surgical instruments for use in a liquid environment have been described as including an evacuation tube wall constructed and positioned to provide a jet-deflecting surface upon which a liquid jet impinges, and while certain embodiments of the inventive liquid-jet surgical instruments for use in a gaseous environment have been described as providing an evacuation tube that is essentially straight and does not include a wall providing a jet-deflecting surface, the liquid jet surgical instruments within the scope of the present invention are not so limited. Specifically, configurations such as those shown in FIGS. 2a and 2b could be employed for a surgical instrument intended for use in a gaseous environment. Similarly, the configurations illustrated in FIGS. 2d and 2e could alternatively be employed for use in a surgical instrument intended to be used in a liquid environment.

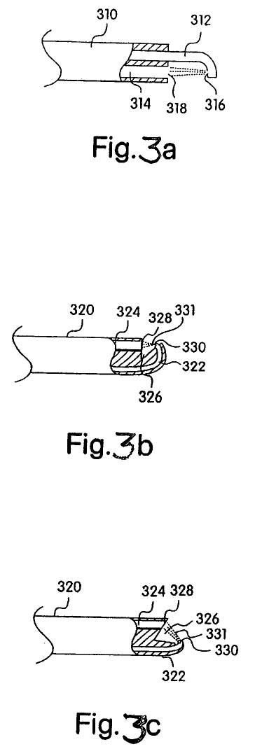

FIG. 3 illustrates a variety of contemplated embodiments for the distal end of a liquid jet surgical instrument, according to the invention. FIG. 3a shows a partially cutaway view of the distal end of the surgical instrument having a sheath 310 from which a pressure tube 312 extends distally. Evacuation lumen 314 is completely contained within and surrounded by sheath 310. During operation, a liquid jet is emitted from jet opening 316 and directed into jet-receiving opening 318 such that at least a central portion of the liquid jet is directed proximally and parallel to a longitudinal axis of sheath 310.

FIGS. 3b and 3c show two embodiments of the distal end of a surgical liquid jet instrument according to the invention where the distal end of sheath 320 essentially completely surrounds both pressure lumen 322 and evacuation lumen 324. The liquid jet path length is created in the instruments by providing a notch 326 at the distal end of sheath 320 where the proximal surface of notch 326 includes a jet-receiving opening 328, and the distal end of notch 326 includes a jet opening 330. In some embodiments, sheath 320 may be constructed from a flexible material, such as a polymeric material. The configuration shown in FIGS. 3b and 3c are substantially similar except that in FIG. 3b, the liquid jet 331 emitted from jet opening 330 has a central region directed proximally and parallel to the longitudinal axis of sheath 320, and in contrast, the central region of the liquid jet 331 for the configuration shown in FIG. 3c, is directed towards jet-receiving opening 328 at an angle of about 45 degrees with respect to the longitudinal axis of sheath 320.

The invention also provides various embodiments of surgical liquid jet instruments having distal ends for insertion into a surgical operating space that have a selected contour and size that are selected to facilitate inserting the distal end into the confined surgical operating space. Certain embodiments of such surgical instruments provided by the invention may include mechanisms for creating relative motion between the pressure tube and evacuation tube in order to change the orientation, positioning, and/or configuration of the tubes with respect to each other, for example to increase a separation distance between the jet opening and the jet-receiving opening. Embodiments of surgical liquid jet instruments having actuating mechanisms, as provided by the invention, can enable the distal end of the instruments to be inserted into a surgical operating space in an undeployed configuration, and subsequently deployed by an operator to provide a desired separation distance between the jet opening and the jet-receiving opening in order to yield a desired liquid jet path length. Embodiments involving deployable liquid jet surgical instruments may be directed to surgical applications involving confined regions within the body of a patient, such as joint capsules or intervertebral discs. In certain such embodiments, the surgical environment surrounding the distal end of the instrument, when it is in operation, is a liquid environment. Such embodiments are described in further detail in commonly-owned U.S. Pat. No. 6,375,635, which is herein incorporated by reference in its entirety.

One aspect of the invention involves the discovery that certain problems may arise when certain conventional surgical liquid jet instruments are used in surgical procedures, especially in a confined space within the body. For example, when an instrument is designed for use within a confined body space, the dimensions of components at the distal end of the instrument may be selected to be small to enable the instrument to fit into the confined space. When dimensions are reduced, the thickness of some components of the instrument, e.g. the wall thicknesses of evacuation and/or pressure tubes, may also be reduced, causing the instrument to present sharp and/or rough edges, which may damage the surrounding tissue when the instrument is inserted into the body.

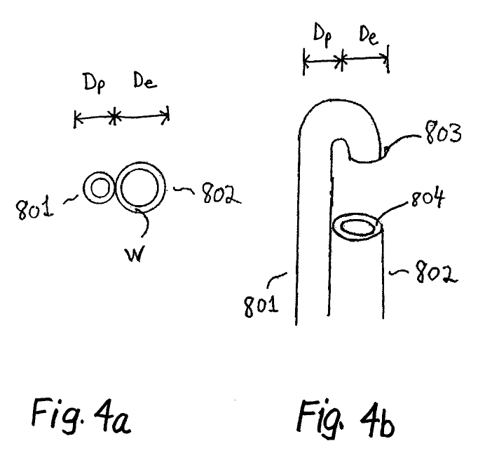

To further demonstrate an example of this problem, FIGS. 4a and 4b illustrate cross-sectional and perspective views, respectively, of one embodiment of a distal end of a conventional surgical liquid jet instrument which is designed for use in confined spaces in the body. This instrument includes a pressure tube 801 and an evacuation tube 802, and although not visible in these figures, as discussed above, a jet nozzle is also provided in the terminal tip 803 of the pressure tube. The outer diameter De of the evacuation tube illustrated in FIGS. 4a and 4b may be, for example, about 0.030-0.080 inch (30-80 mils; 0.75-2.00 millimeters), and the outer diameter of the pressure tube may be about 20-40 mils (0.5-1.0 mm). The wall thickness W of either or each of the tubes may be, for example, about 0.002 inch-about 0.016 inch, and in one embodiment the wall thickness is about 0.004 inch-about 0.008 inch, and in another embodiment, the wall thickness of at least one of the tubes is about 0.005 inch (0.125 mm). For example, an embodiment using a tube having a wall thickness of about 0.005 inch (0.125 mm) may be sized for use within confined spaces, however, the tube wall thickness at the terminal tip of each tube may be comparable to the thickness at the edge of a dull knife. Therefore, movement of this instrument within the body may lead to inadvertent scraping, cutting, gouging, etc. of tissue. In some embodiments, this may cause undesirable damage to surrounding tissue. Furthermore, in some embodiments, it is also desirable to reduce the irritation and tissue damage which may be caused by movement of the instrument within the body, even when the instrument is not used within a confined space.

To prevent and/or reduce trauma to the surrounding tissue during surgical procedures utilizing liquid jet instruments, in certain embodiments of the invention, the terminal tip of the wall of the evacuation tube and/or the pressure tube is modified to reduce exposure of surrounding tissue to sharp corners or edges that could gall or cut tissue undesirably and that may be present on surgical liquid jet instruments manufactured with an essentially planar cut made perpendicular to the center axis of the evacuation/pressure tube at the terminal tip. As mentioned above, forming the terminal tip of the wall of the evacuation tube and/or the pressure tube with a planar cut perpendicular to the center axis of the lumen at the terminal tip can leave edges of the terminal tip of the wall of the tube that may inadvertently damage and/or cut tissue regions where it is not desirable. Accordingly, in certain embodiments of the invention, the walls of either or both of the evacuation tube and the pressure tube are provided with a blunted terminal tip. “Blunted” as defined previously is meant to encompass any treatment in which the terminal tip of a tube is less sharp in comparison to the terminal tip of an untreated tube with a planar cut perpendicular to the center axis of the tube at the terminal tip to the extent necessary so as to be substantially non-traumatic to tissue against which the terminal tip(s) of the tube(s) may be brought into contact during normal usage for procedures for which the instrument is indicated. As discussed in further detail below, in one embodiment, the blunted terminal tip of the tube wall may be formed by smoothly curving or bending the edges of the walls inwardly or outwardly with respect to the center axis of the tube at its terminal tip. In other embodiments, the blunted terminal tip may be formed by mechanically or chemically altering the terminal tip of the tube wall, and/or with heat treatment. In yet other embodiments, the blunted terminal tip may be formed by an attachment secured to the terminal tip of the tube wall.

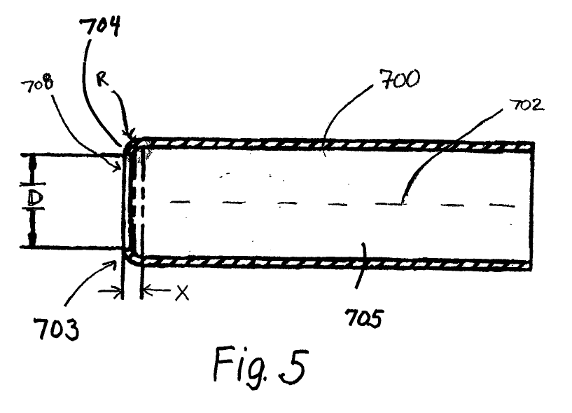

As illustrated in the embodiment shown in FIG. 5, the wall 700 of the illustrated evacuation tube has a blunted terminal tip 703 formed by an inwardly curved wall portion 704 at the terminal tip adjacent the jet-receiving opening 708. The curved wall portion 704 at the terminal tip of the evacuation tube curves in towards the center axis 702 of the evacuation lumen at the terminal tip, which in the illustrated embodiment also is collinear with the longitudinal axis of evacuation lumen 705, so to minimize trauma to the tissue surrounding the tissue regions where cutting and/or ablation is desirable. In one embodiment, the radius of curvature R of the blunted terminal tip 703 of curved wall portion 704 of the evacuation tube is uniform about the circumference of the tube at the terminal tip. However, in other embodiments, the radius of curvature may vary about the periphery of the terminal tip. In one particular embodiment, where the outside diameter of the tube 700 forming the evacuation lumen is about 0.072″, and the inside diameter of the tube 700 forming the evacuation lumen is about 0.063″, the length X of the portion 704 of the wall of the evacuation tube that is curved is about 0.010″, and the curved portion 704 of the wall has a uniform radius of curvature R of about 0.010″ around the circumference of the tube 700 forming evacuation lumen. In this embodiment, the resulting jet-receiving opening diameter D is about 0.053″. As shown in FIG. 5, with the above-described blunted terminal tip of the evacuation tube wall, the diameter of the jet-receiving opening is smaller than the diameter of the lumen. When the curved portion 704 at the terminal tip of the evacuation tube 700 has a uniform radius of curvature, the diameter of the jet-receiving opening 708 typically is equal to the inside diameter of the lumen less the inwardly extending portion of the wall.

It should be appreciated that although FIG. 5, as well as other figures, are described with respect to an evacuation tube, in other embodiments, the terminal tips of other tubular components of a surgical liquid jet instrument, such as for example, the pressure tube may be blunted. It should be understood that when a reference is made in this application to an evacuation tube or any other type of tube with a blunted terminal tip, that in certain embodiments, such reference may encompass any other tubular component on a surgical liquid jet instrument having a blunted terminal tip, such as for example, a pressure tube.

The blunted terminal tip 703 of the tube illustrated in FIG. 5 may be manufactured using a variety of techniques, and the present invention is not limited in this respect. For example, in one embodiment, the terminal tip of the tube may be hand polished to obtain a desired smoothness or curvature. In another embodiment, swaging may be used to curve in the terminal tip of the tube. Other known processes for rounding and/or partially closing an end of a tube, as well as processes relating to the formation of a “bullet nose” configuration may also be used. In another embodiment, the terminal tip of the tube may be molded into the desired curvature. In yet another embodiment, the desired terminal tip curvature of the tube may be provided by utilizing a “cupping” method.

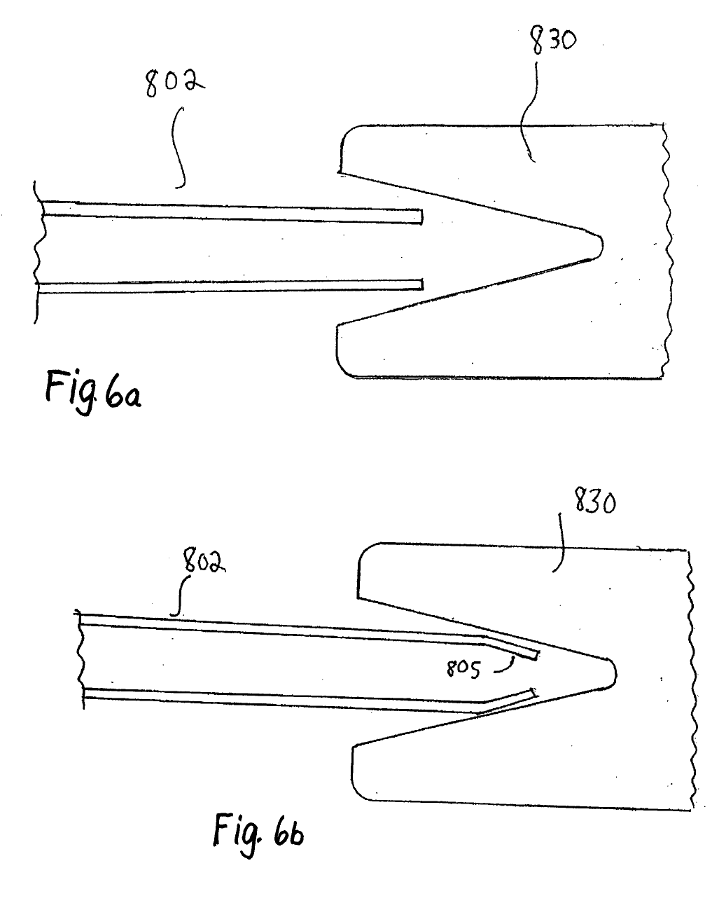

A representative example of a “cupping” method that may be employed in certain embodiments of the invention is illustrated in FIGS. 6a and 6b. A length of stock tubing 802 is cut to a desired length. As shown in FIG. 6a, a cup-shaped mold, or cupping device 830, having an inside contour similar to the desired curvature of the terminal tip of the tube, in the illustrated embodiment an evacuation tube, is provided. The cupping device is positioned adjacent the terminal tip of the evacuation tube, and with the application of pressure and/or heat, the desired “cup” configuration is formed. In one embodiment, the cupping device 830 rotates and the terminal tip of the evacuation tube is placed within the cupping device 830 to a defined depth to obtain the desired curvature. For example, a cupping device 830, may be placed in the chuck of a lathe or other rotating machine, or otherwise caused to rotate, and the terminal tip of tubing 802 bends to the curvature of the cupping device 830 and may become narrower in a terminal tip region 805. It should be appreciated that in some embodiments, the cupping device may increase the thickness of regions of the tube walls. It should be appreciated that although a “cup-shape” may be desirable for circular cross-sections, similar “cupping” methods may be implemented to bend the terminal tip of the evacuation tube into the jet-receiving opening for non-circular cross-sections as well. The term “cupping” is understood to encompass any method which curves or otherwise deflects the outer surface of a tube's terminal tip inwardly towards its center axis. As mentioned above, in this particular embodiment, the tubing 802 forms an evacuation lumen, however, this invention also contemplates performing the “cupping” method on other tubular components of a surgical liquid jet instrument, such as a pressure tube.

Another representative example of a “cupping” method that may be employed in certain embodiments of the invention involves a mandrel or pin inserted into the tube, where the mandrel may help to define the curvature at the terminal tip of the tube. The mandrel and the tube may both be placed in the chuck of a lathe or other rotating machine, or otherwise caused to rotate. Then, a cupping device, which may simply be a piece of slanted or curved material, such as a metal, is pressed against the rotating tube to bend it against the mandrel to form the curvature of the terminal tip of the tube. After the desired contour is achieved, the mandrel is removed from the tube.

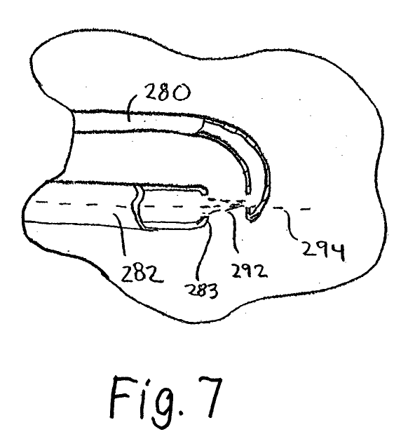

The inwardly angled/curved terminal tip modification of the evacuation tube may be configured to be functionally essentially the same as the above described evacuation tube embodiments. Therefore, it may be implemented in essentially any of the above described surgical liquid jet instrument embodiments. For example, FIG. 7 illustrates a portion of the distal end of a surgical liquid jet instrument similar to the embodiment disclosed in FIG. 2d, except that the wall 282 of the evacuation tube has a radius of curvature at its terminal tip 283 such that the wall 282 of the evacuation tube has a blunted distal terminal tip. In particular, the terminal tip of the evacuation tube wall protrudes inwardly toward the center axis 294 of the evacuation tube at the terminal tip.

Further, as described above and illustrated in FIG. 2e, it may be desirable for the wall of the evacuation tube to have a constriction 302 located proximal to the jet-receiving opening to act as a venturi. In one embodiment, the constriction 302 may be formed during the “cupping” process which forms the desired terminal tip inward curvature of the wall of the evacuation tube. For example, a mold, including both the shape of the constriction and the radius of curvature, may be placed up against the distal end of the wall of the evacuation lumen to form both the venturi constriction and the blunted terminal tip.

The inventive surgical liquid jet instruments having a blunted terminal tip on components of the instrument, such as the evacuation tube wall or pressure tube wall may be well suited for insertion into the spine of a patient for spinal surgery applications. The spinal column is made up of the vertebrae bones which are connected in the anterior (front) portion of the spine by intervertebral discs. The intervertebral discs provide support and cushioning to the spine, serving as the spine's shock absorbing system. The discs also allow for some spinal motion, although individual disc movement is very limited. Many ligaments and muscles are also attached to the posterior (back) portion of the spine to provide power for spine movement. Each intervertebral disc is composed of an outer ring-like component made up of concentric sheets of collagen fibers, called the annulus fibrosus, and an inner semi-gelatinous tissue, called the nucleus pulposus. The radial structure of the annulus fibrosus prevents the nucleus pulposus from protruding from the disc. In the spinal column, there are four segments of spinal curvatures. From the superior (top) to the inferior (bottom) portions of the spinal column, these curvatures include the cervical, thoracic, lumbar, and sacral portions.

Surgical procedures on intervertebral discs in the lumbar, cervical, or thoracic portions of the spine are performed for a variety of reasons, which include treatment of tears in the annulus fibrosus, herniation of the nucleus pulposus, and significant disc height loss. Herniation results when the annulus fibrosus weakens such that the soft central nucleus pulposus bulges through the layers of the annulus fibrosus. The nucleus pulposus may bulge or leak posteriorly towards the spinal cord and major nerve roots, causing significant pain and discomfort.

One of the most common surgical procedures for treating a disc herniation is a discectomy. This procedure involves the removal of portions of the disc which impinge on the nerve roots or spinal cord posterior to the disc. All or portions of the nucleus pulposus may be removed to minimize the risk of additional herniations. The nucleus pulposus may be accessed by a variety of recognized surgical techniques. In certain embodiments, the nucleus pulposus is accessed directly through the annulus fibrosus. For example, the nucleus pulposus may be accessed by an incision through either the anterior portion or the posterior portion of the annulus fibrosus. In other embodiments, where an opening has already formed within the annulus fibrosus, it may be desirable to access the nucleus pulposus through this opening. In yet other embodiments, the nucleus pulposus is accessed via the vertebral body or through an end plate. For example, in certain embodiments, the nucleus pulposus may be accessed by penetrating into the spinal column through the sacral portion. It should be appreciated that in certain embodiments, the inventive surgical instruments may be inserted into the spine using a variety of techniques known for entering the spine, as would be recognized by one skilled in the art.

Various devices may be used to replace portions of the removed nucleus pulposus and/or annulus fibrosus, or the disc entirely. For example, when only the nucleus pulposus is replaced, a prosthetic device may be inserted through a hole created in the annulus fibrosus. Once the prosthetic device is within the confines of the annulus fibrosus, the device may expand, inflate, or deploy to fill the area of the disc that was removed.

In certain surgical applications, it may be desirable to remove all or portions of the inner nucleus pulposus, leaving the annulus fibrosus as intact as possible. However, conventional surgical instruments that are used to remove portions of the intervertebral disc suffer from not being able to distinguish between the two components of an intervertebral disc. As mentioned previously, because the inventive surgical liquid jet instruments can be configured and operated to provide for selective tissue differentiation in cutting and removal, they may, according to certain embodiments of the invention, be advantageously utilized in surgical procedures to remove all or portions of the inner nucleus pulposus while leaving the annulus fibrosus and/or other portions of the spine, such as the cartilage of the end plates, as intact as possible. Moreover, in certain embodiments, surgical liquid jet instruments including the inventive blunted terminal tip of the evacuation and or pressure tube wall may be used advantageously for such applications. If surgical liquid jet instruments lacking such blunted terminal tips are used to attempt to remove only the nucleus pulposus, portions of such a surgical instruments comprising sharp edges that abut either the annulus fibrosus or surrounding cartilage may tend to cause injury to such tissue due to the sharp corners/edges at the periphery of the terminal tip of either the evacuation lumen wall or the pressure lumen wall pressing against such tissue during use. Consequently, portions of the annulus fibrosus and/or surrounding cartilage may be undesirably cut or damaged. This may be compounded by the fact that there may be limited visibility when performing procedures within the intervertebral discs. Not only may this contribute to additional post-surgical scar tissue, added pain, and a longer recovery time, but the annulus fibrosus has been shown to have a very limited healing capacity. Healing of the annulus fibrosus typically results in the formation of a thin fibrous film along the perimeter of the annulus fibrosus that does not ever reach the original strength of the annulus pulposus.

However, in certain embodiments of the present invention, a surgical liquid jet instrument is provided with a modified evacuation tube wall and/or a modified pressure tube wall providing a blunted terminal tip, which may be used in inventive spinal surgical procedures to remove the nucleus pulposus without injuring, or with reduced injury to, the surrounding annulus fibrosus. When the terminal tip of the evacuation tube wall and/or the pressure tube wall is blunted, undesirable trauma to the intervertebral disc may be reduced or minimized when the surgical liquid jet instrument is used to remove portions of the disc. When the terminal tip of the evacuation tube wall and/or the pressure tube wall is blunted, the outer edges and surfaces of the tube walls of the instrument will tend to not harm or cause less harm to the surrounding tissue. Therefore, the tissue cutting/ablation/removal can be more readily limited to only the desirable regions which contact the liquid jet.

In certain embodiments, the pressure of the liquid jet may further be selected and/or adjusted to limit trauma to the tissue surrounding the excised tissue. As described above, the nucleus pulposus has a gel-like consistency. In contrast, the annulus fibrous is a much more rigid collagen lamellae structure. Therefore, one can adjust the pressure of the liquid jet to be sufficient to cut and ablate the nucleus pulposus, while not sufficient to damage the more rigid annulus fibrosus.

In certain embodiments of the invention, a surgical liquid jet instrument is employed for use in a surgical method involving the cutting or ablating of a first tissue within the spine of a patient, for example the nucleus pulposus within the intervertebral disc of a patient, while not cutting or ablating an underlying, adjacent, surrounding, and/or interdigitating tissue, e.g. the annulus fibrous, desired to be preserved from damage. An exemplary method comprises use of a surgical liquid jet instrument having a blunted terminal tip of the evacuation tube wall and/or the pressure tube wall for such a procedure.

For example, in one embodiment, the terminal tip of the evacuation lumen wall is blunted such that the outer surface of the terminal tip of the evacuation lumen wall curves/angles inwardly towards the jet-receiving opening, as provided by the invention, e.g. such as illustrated in FIGS. 5-7 and as described above, and, optionally, the instrument has a distal end specifically designed for performing surgical procedure in the intervertebral disc of the patient. In certain spinal applications, instruments with smaller sized distal ends may be preferred to minimize the size of the opening into the disc required for access, which may be, for example, through the annulus fibrosus. In certain embodiments, such as for those instruments specifically designed for spinal applications, the outer diameter of the evacuation tube may range from about 0.5 mm-about 2 mm, and the outer diameter of distal end of the instrument, including the evacuation tube combined with the pressure tube may range from about 0.8 mm-about 3 mm. Upon insertion of the instrument into the disc, the operator of the instrument can then, optionally and for embodiments involving deployable distal ends with variable jet lengths, deploy the distal end of the instrument to create a separation distance between the jet opening and the jet-receiving opening, defining a liquid jet path length. The operator can then turn on a pump or dispenser supplying high pressure liquid to the device, as discussed previously, in order to create a liquid jet with the surgical instrument. The liquid jet can then be directed towards the jet receiving opening of the evacuation tube of the instrument, and will tend to create an entrainment region surrounding the liquid jet, which can be effective for cutting or ablating a selected tissue within the intervertebral disc.

The method may include varying and/or selecting the pressure of the liquid jet to be sufficient to cut and/or ablate portions of the nucleus pulposus, yet not high enough to damage the surrounding annulus fibrosus. In certain embodiments, the pressure required to cut and/or ablate portions of the nucleus pulposus, while not damaging the surrounding annulus fibrosus may be dependent upon several factors. For example, this pressure may depend on the extent to which the annulus fibrosus has stiffened, and/or the nucleus pulposus has dehydrated, both of which generally increase with age. This pressure may also be dependent upon whether abnormal calcification is present. In certain embodiments, a pressure between about 2,000 psig and about 15,000 psig will provide a sufficient degree of differentiation to cut and/or ablate portions of the nucleus pulposus without damaging the healthy portions of the annulus fibrosus. However, this value may vary based upon the condition of the patient's spine, and on the configuration of the particular surgical instrument. A skilled operator of the surgical instrument could readily determine a more specific desirable pressure range. More particularly, by making adjustments to the pressure, such as by increasing the pressure until reaching a pressure that cuts and/or ablates the nucleus pulposus.

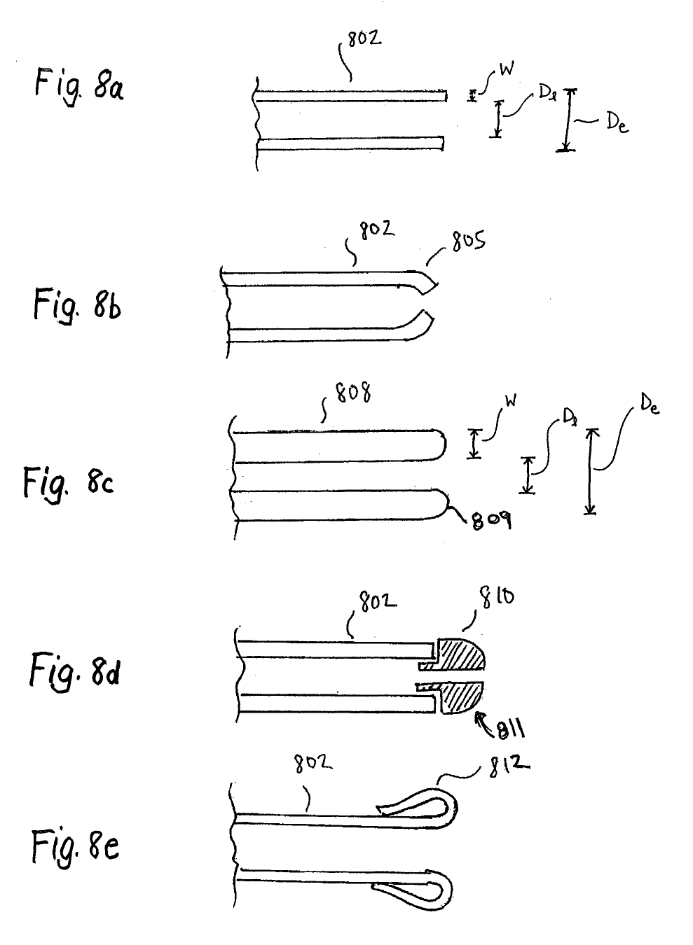

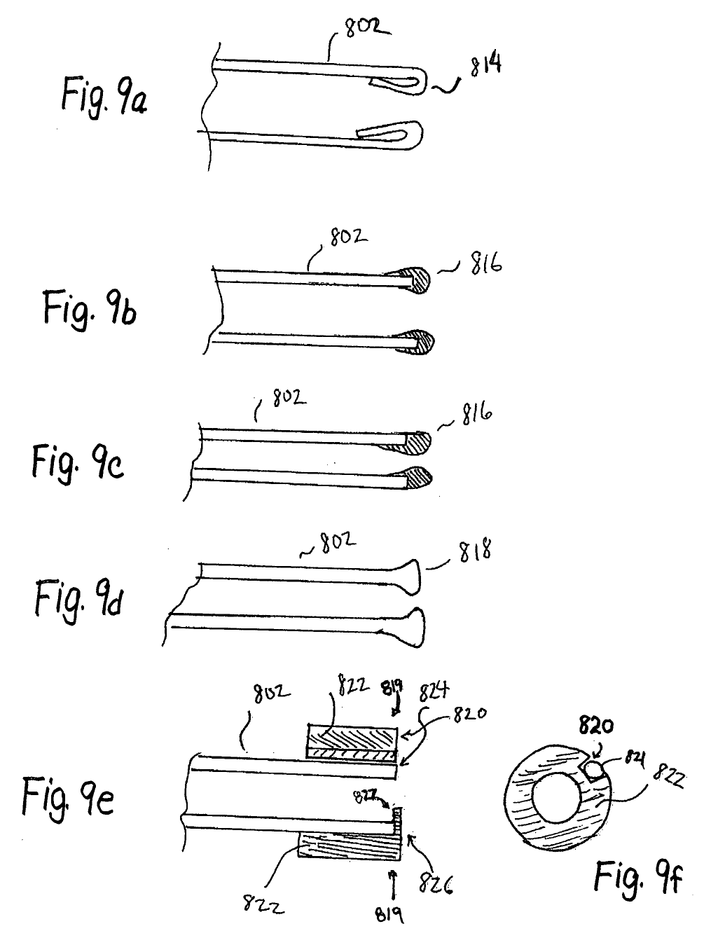

The above described cupping method is one method for forming a liquid jet surgical instrument which minimizes or eliminates the tendency of a terminal tip of a tube, and in particular a tube with a small thickness, to gouge tissue. However, several other treatments or procedures for blunting the terminal tip of the tubes used to construct surgical liquid jet instruments may alternatively be used in the context of the present invention. FIGS. 8 and 9 show some representative embodiments of such terminal tip blunting treatments or procedures as taught by the present invention. As discussed above, although the following figures refer to an evacuation tube, it should be appreciated that these treatments or procedures for blunting the terminal tip of a tube are also applicable for other tubular components of the instrument, such as the pressure tube. For example, FIG. 8a shows an evacuation tube 802, with an outer diameter De of about 0.040″ (1.0 mm), an inner lumen diameter Dl of about 0.030″ (0.76 mm), and a wall thickness W of about 0.005″ (0.125 mm). FIG. 8a is representative of a tube wall with a terminal tip having edges formed from a planar cut perpendicular to the center axis of the lumen, which is typical of conventional liquid jet surgical instrument tubes. In contrast, FIG. 8b shows an inventive evacuation tube 802 after being cupped at the terminal tip as described above, such that it has a blunted terminal tip 805.

Another approach to forming a blunted terminal tip is shown in FIG. 8c. In this embodiment, the lumen is formed from tubing 808 which has a rounded terminal tip end surface 809, and has a substantially larger wall thickness in comparison to the tube shown in FIG. 8a. In contrast to the tube of FIG. 8a, the tubing 808 of FIG. 8c has a wall with a thickness approximately three times as thick (15 mils, 0.375 mm), and has a larger exterior diameter De (50 mils, 1.25 mm), but a smaller lumen diameter Dl (20 mils, 0.5 mm). This particular embodiment of a blunted terminal tip may help to reduce the amount of tissue damage in comparison to the configuration shown in FIG. 8a. However, with the smaller lumen diameter, it will likely have greater backpressure and a larger outer profile.

As mentioned above, another approach to forming a blunted terminal tip is to secure an attachment to the terminal tip of the tube walls. For example, as shown in FIG. 8d, a grommet 810 is fitted to the terminal tip of tube 802. In this embodiment, the grommet 810 has a rounded outer edge which forms the blunted terminal tip 811 of the illustrated evacuation tube. In FIG. 8d, grommet 810, is shown as loose-fitting for clarity, but it should be appreciated that the grommet 810 would, in practice, be configured to tightly fit into the lumen of tube 802, optionally bonded by adhesive, welding, swaging or other permanent bonding procedure.

As mentioned above, the blunted terminal tip of the tube may be formed by smoothly curving or bending the edges of the tube 802 walls inwardly or outwardly with respect to the center axis of the lumen at the terminal tip. FIG. 8e illustrates one embodiment where the edges of the tube walls are curved outwardly with respect to the center axis of the lumen. In this respect, the tube wall in the terminal tip region 812 is flared and bent back to form a smooth termination. To bend back this terminal tip region 812 of the tube around the entire perimeter of the tube, the terminal tip region may be cut into strips or pieces which may be bent back onto the outer surface of the tube. To assist in the formation of this blunted terminal tip, the terminal tip region 812 may also be softened by heating. Although the profile of this embodiment is larger, and there may be some Venturi effect, the tissue damage or gouging caused by the contact of the terminal tip of the tube 802 and the body may be significantly reduced.

Turning to FIGS. 9a-9e, several additional representative embodiments of a blunted terminal tip of an evacuation tube 802 are also illustrated. For example, FIG. 9a shows a tube 802 having a terminal tip region 814 which is bent into the tube. As discussed above with respect to the embodiment in FIG. 8e, this terminal tip region 814 may also be cut into strips or pieces around the perimeter of the terminal tip of the tube to assist in bending the wall in the terminal tip region into the lumen. Also, to assist in the formation of this blunted terminal tip, the terminal tip region 814 may be softened by heating.

Furthermore, FIGS. 9b and 9c show an evacuation tube 802 where the terminal tip of the tube has been coated by, for example, dipping in a molten or fluid material, which is then set or dried, to form a blunted terminal tip 816. Any of a variety of materials can be used to obtain this effect, depending on the operating pressure and the composition of the tubing. Such representative materials include molten metal, such as a medically-approved solder or a low-melting non-toxic metal or metal alloy; molten ceramic; and, if the liquid jet beam of an instrument in which such an evacuation tube configuration is employed is well focused and does not contact the evacuation lumen at its terminal tip, polymeric materials. Polymers having high tensile strength may be preferred, such as PEEK and polyaramids. Precise control of the size and shape of the blunted distal tip 816 is possible by control of the temperature of the tubing and of the liquid material, and when relevant, of the concentration and composition of the molten or fluid material used to form the coating. The embodiment disclosed in FIG. 9c is similar to the embodiment shown in FIG. 9b except that portions of the material forming the blunted terminal tip 816 have been removed from the outer periphery of the evacuation tube 802 in FIG. 9c, to minimize the total outer diameter while maintaining the blunted terminal tip and Venturi effects.

In certain embodiments, portions of the material forming the blunted terminal tip may also be removed from the inner periphery of the evacuation tube 802. For example, portions of the terminal tip, such as the inside portion of the terminal tip 805 illustrated in FIG. 8b may be removed. In this embodiment, the Venturi effects may be minimized. The material may be removed in a variety of methods, such as by reaming, drilling, or grinding down the material. In certain embodiments, enough material is removed such that the inner diameter of the tube 802 at its terminal tip is similar to the size it was before the terminal tip was blunted.

FIG. 9d illustrates yet another approach to forming a blunted terminal tip of an evacuation tube 802. In this embodiment, the tube has been blunted at its terminal tip 818, to form a less aggressive edge, by a mechanical force, such as hammering. To assist in the formation of this blunted terminal tip, the metal of the tube may be softened by heating.

FIGS. 9e and 9f show the formation of a blunted terminal tip 819 of an evacuation tube 802 by the addition of an attachment, such as an external collar 822. As shown in more detail in FIG. 9f, a notch 820 may be formed in the collar 822 for accommodating the pressure tube 821. The distal end of the collar may be flush with the distal end of the evacuation tube as shown at 824, or it may overhang the distal end of the tube as shown at 826. Furthermore, the collar may extend into the lumen of the evacuation tube 802, as shown at 827 to create a Venturi effect, if desired.

While several embodiments of the invention have been described and illustrated herein, those of ordinary skill in the art will readily envision a variety of other means and structures for performing the functions and/or obtaining the results or advantages described herein, and each of such variations, modifications and improvements is deemed to be within the scope of the present invention. More generally, those skilled in the art would readily appreciate that all parameters, dimensions, materials, and configurations described herein are meant to be exemplary and that actual parameters, dimensions, materials, and configurations will depend upon specific applications for which the teachings of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described. The present invention is directed to each individual feature, system, material and/or method described herein. In addition, any combination of two or more such features, systems, materials and/or methods, provided that such features, systems, materials and/or methods are not mutually inconsistent, is included within the scope of the present invention.

In the claims (as well as in the specification above), all transitional phrases or phrases of inclusion, such as “comprising,” “including,” “carrying,” “having,” “containing,” “composed of,” “made of,” “formed of,” “involving” and the like shall be interpreted to be open-ended, i.e. to mean “including but not limited to” and, therefore, encompassing the items listed thereafter and equivalents thereof as well as additional items. Only the transitional phrases or phrases of inclusion “consisting of” and “consisting essentially of” are to be interpreted as closed or semi-closed phrases, respectively. The indefinite articles “a” and “an,” as used herein in the specification and in the claims, unless clearly indicated to the contrary, should be understood to mean “at least one.”

What is claimed is:

1. A device comprising:
a surgical instrument having a distal end adapted to perform a surgical procedure on a patient and a proximal end adapted to be controllable by an operator, the instrument including:
a pressure tube comprising a pressure lumen defined by a wall of the pressure tube, the pressure tube having sufficient burst strength to conduct a high pressure liquid towards the distal end of the instrument, the pressure tube including at least one nozzle providing a jet opening; and
an evacuation tube comprising an evacuation lumen defined by a wall of the evacuation tube, the evacuation lumen including a jet-receiving opening locatable opposite the jet opening to receive a liquid jet when the instrument is in operation;
the nozzle being shaped to form the liquid jet as a liquid at high pressure flows therethrough; and
wherein the evacuation tube wall has a blunted terminal tip.

2. A device as in claim 1, wherein the maximum cross-sectional thickness of the blunted terminal tip of the wall is greater than the thickness of portions of that wall proximal to the blunted terminal tip.

3. A device as in claim 1, wherein the maximum cross-sectional thickness of the blunted terminal tip of the wall is the thickest portion of that wall.

4. A device as in claim 1, wherein the evacuation tube is non-integrally formed with the pressure tube.

5. A device as in claim 1, wherein the terminal tip of the evacuation tube wall has a perimeter, and wherein the evacuation tube wall is blunted around a majority of its perimeter.

6. A device as in claim 1, wherein the evacuation tube has a center axis at the terminal tip, and wherein the terminal tip of the wall of the evacuation tube is curved and/or is angled inwardly towards the center axis to form the blunted terminal tip.

7. A device as in claim 1, wherein the evacuation tube has a center axis at the terminal tip, and wherein the terminal tip of the wall of the evacuation tube is curved and/or is angled outwardly away from the center axis to form the blunted terminal tip.

8. A device as in claim 1, wherein the terminal tip of the wall of the evacuation tube is treated with heat to at least partially melt and/or soften the wall to decrease the radius of curvature and/or sharpness of the edges of the wall prior to heat treatment so as to form the blunted terminal tip.

9. A device as in claim 1, wherein the terminal tip of the wall of the evacuation tube is treated with a coating to decrease the radius of curvature and/or sharpness of the edges of the wall prior to coating so as to form the blunted terminal tip.

10. A device as in claim 1, wherein the terminal tip of the wall of the evacuation tube is mechanically altered to decrease the radius of curvature and/or sharpness of the edges of the wall prior to mechanical alteration so as to form the blunted terminal tip.

11. A device as in claim 1, further comprising an attachment secured to the terminal end of the evacuation tube, wherein the attachment forms the blunted terminal tip.

12. A device as in claim 1, wherein the liquid at high pressure is supplied to the jet opening when the instrument is in operation at a pressure of at least 1,000 psig.

13.-16. (canceled)

17. A device as in claim 1, wherein the maximum cross-sectional thickness of the evacuation tube wall is between about 0.002 inches and about 0.016 inches in a region of the blunted terminal tip.

18. A device as in claim 1, wherein both the evacuation tube wall and the pressure tube wall have blunted terminal tips.

19. A device as in claim 1, wherein the distal end of the surgical instrument has a shape and size selected to facilitate insertion of the distal end into a region of the body of the patient defining a surgical site.

20. A device as in claim 19, wherein the region of the body of the patient defining a surgical site is the spine of the patient.

21. A device as in claim 1, wherein the surgical instrument is constructed and arranged to be inserted into the annulus fibrosus of an intervertebral disc.

22. A device comprising:
a surgical instrument having a distal end adapted to perform a surgical procedure on a patient and a proximal end adapted to be controllable by an operator, the instrument including:
a pressure tube comprising a pressure lumen defined by a wall of the pressure tube, the pressure tube having sufficient burst strength to conduct a high pressure liquid towards the distal end of the instrument, the pressure tube including at least one nozzle providing a jet opening;
an evacuation tube comprising an evacuation lumen defined by a wall of the evacuation tube, the evacuation tube including a jet-receiving opening locatable opposite the jet opening to receive a liquid jet when the instrument is in operation;
the nozzle being shaped to form a liquid jet as a liquid at high pressure flows therethrough; and
a terminal tip of the evacuation tube having a center axis and a perimeter, wherein the terminal tip of the evacuation tube wall is curved and/or is angled inwardly towards the center axis around a majority of the perimeter of the evacuation tube.

23.-27. (canceled)

28. A method comprising:
inserting a surgical liquid-jet instrument into a surgical site in the body of a patient;
creating a liquid jet with the surgical liquid-jet instrument;
directing the liquid jet towards a jet-receiving opening of an evacuation tube of the surgical liquid-jet instrument, wherein the evacuation tube wall has a blunted terminal tip; and
cutting or ablating a selected tissue within the surgical site with the liquid jet.

29.-35. (canceled)

36. A method comprising:
inserting a surgical liquid jet instrument into the spine of a patient; and
cutting, ablating, and/or removing with a liquid jet of the instrument a first tissue within the spine while not cutting, ablating, and/or removing with the liquid jet of the instrument a second tissue within the spine.

37.-38. (canceled)

39. A method of manufacturing a surgical liquid jet instrument comprising a pressure tube and an evacuation tube, the method comprising:
forming a blunted terminal tip of an evacuation tube wall of the surgical liquid jet instrument; wherein
the pressure tube comprises a pressure lumen defined by a wall of the pressure tube, the pressure tube having sufficient burst strength to conduct a high pressure liquid towards a distal end of the instrument, the pressure tube including at least one nozzle providing a jet opening, wherein the nozzle is shaped to form a liquid jet as a liquid at high pressure flows therethrough; and wherein
the evacuation tube comprises an evacuation lumen defined by a wall of the evacuation tube, the evacuation tube including a jet-receiving opening having a cross-sectional area and locatable opposite the jet opening.

40.-43. (canceled)
